(19) United States
(12) Patent Application Publication
(10) Pub. No.: US 2010/0260687 A1
(43) Pub. Date: Oct. 14, 2010

(54) PERSONAL CARE COMPOSITIONS INCLUDING POLYURETHANE DISPERSIONS

(76) Inventors: Wei H. Yu, Edison, NJ (US); Susan L. Jordan, Doylestown, PA (US); Wing K. Li, East Brunswick, NJ (US); Debkumar Bhattacharjee, Lake jackson, TX (US)

Correspondence Address: The Dow Chemical Company, P.O. BOX 1967, Midland, MI, 48641, US

(21) Appl. No.: 12/679,735

(22) PCT Filed: Sep. 23, 2008
(86) PCT No.: PCT/US08/77341

§ 371 Date: May 21, 2010

Related U.S. Application Data
(60) Provisional application No. 60/974,646, filed on Sep. 24, 2007.

(51) Int. Cl. A61K 8/87; A61Q 1/10; A61Q 1/06; A61Q 1/08; A61Q 3/02; A61Q 5/06; A61Q 5/12; A61Q 9/04; A61Q 15/00; A61Q 17/00; A61Q 17/02; A61Q 17/04; A61Q 19/00; A61Q 1/02; A61P 31/00; A61P 17/00

(52) U.S. Cl. 424/43; 424/61; 424/63; 424/59; 424/70.11; 424/65; 424/62

(57) ABSTRACT

Personal care compositions comprising a high solid aqueous polyurethane dispersion, substantially free of organic solvent and tertiary amines, are described.

### BACKGROUND

Personal care compositions, such as for skin care, nail care, or hair care, often incorporate resins to provide moisture resistance, luster, and spreadability.

Polyurethanes exhibit excellent properties, but are difficult to incorporate into personal care compositions. Polyurethanes are generally hydrophobic, and thus not readily water dispersible. In some cases, anionic groups, such as carboxylic acids, have been incorporated into the prepolymer to aid in water dispersibility. This requires neutralization of the resulting dispersion, usually by a tertiary amine. Some polyurethane dispersions used in personal care compositions do contain tertiary amine, for example, AVALURE UR 450, from Noveon, however, because tertiary amines are unpleasant smelling and are low molecular weight volatile organic compounds, the industry has sought for other options. Additionally, many polyurethane dispersions require solvents during manufacturing that are considered toxic by some governmental groups, such as N-methylpyrrolidone (NMP), which has been the subject of a Health Hazard Advisory in the state of California.

Thus what is needed are solvent free and amine free aqueous polyurethane dispersions and methods for use in personal care compositions.

### SUMMARY

In one embodiment, the present invention provides personal care compositions comprising a high solid aqueous polyurethane dispersion, substantially free of organic solvent and tertiary amines.

### DETAILED DESCRIPTION

In one embodiment, the present invention provides personal care compositions comprising a high solid aqueous polyurethane dispersion, substantially free of organic solvent and tertiary amines, and at least one of a cosmetically acceptable emollient, surfactant, emulsifier, preservative, or rheology modifier, including thinners or thickeners.

“Personal care compositions” are those that are to be topically applied to a person. Examples of personal care compositions include skin care products (e.g., facial cream, moisturizers, lotions, sunscreens, foundation, mascara, eye-liner, lipstick, and the like), nail care products (such as polish and conditioners), and hair care products (including styling gels and hairsprays). “High solid” refers to concentrations greater than 30%, preferably greater than 35%, preferably greater than 40%, and more preferably, greater than 45%. “Substantially free” means having a concentration no higher than levels normally found as impurities in ingredients for such compositions. In one embodiment, “substantially free” means concentrations less than 0.1%, preferably less than 0.01%, or more preferably, less than 0.001%. “Cosmetically acceptable” refers to ingredients typically used in personal care compositions, and is intended to underscore that materials that are toxic, irritating, or unpleasant smelling when present in the amounts typically found in personal care compositions are not contemplated as part of the present invention.

In one embodiment, the aqueous polyurethane dispersion is a high solid aqueous polyurethane dispersion, substantially free of tertiary amines, and made under a VOC-free manufacturing process. Methods of making such dispersions can be found at U.S. Pat. No. 5,539,021, U.S. Pat. No. 5,688,42, U.S. Pat. No. 5,959,027, U.S. Pat. No. 6,087,440, and U.S. Pat. No. 6,451,908, the entire disclosures of which are incorporated herein as if reproduced in their entireties. In a preferred embodiment, the aqueous polyurethane dispersion is about 45-60% solid, stabilized by a combination of external anionic and nonionic surfactants, and is obtained from aliphatic isocyanate/polyester polyol based polymers. Preferred aqueous polyurethane dispersions are derived from aliphatic isocyanate, for example, isophorone diisocyanate (IPDI), dicyclohexylmethane diisocyanate (H12MDI), tetramethylxylene diisocyanate (TMXDI), hexamethylene diisocyanate (HDI), 1,3- and 1,4-bis(isocyanatomethyl)cyclohexane, and mixtures thereof and a polyol. The polyol component can be polyether polyols or polyester polyols, or a combination thereof, although polyester polyols are preferred. These components (isocyanate and polyol) are combined to form a prepolymer. A small amount of ethylene oxide moieties may be incorporated into the polymer backbone for steric stabilization as well as for improving dispersibility of the prepolymer. The prepolymer is then chain extended to form the desired polymer, for example, a prepolymer based on the foregoing has % NCO from 0.5 to 15%, and can be extended by adding stoichiometric amounts of water soluble diamines in combination with water.

In a preferred embodiment, the high solid aqueous polyurethane dispersion is produced from an isocyanate terminated, neutral prepolymer prepared from isophorone diisocyanate (IPDI) and polyol blend comprised of an aliphatic polyester polyol, followed by chain extension of the prepolymer with aqueous ethylene diamine. Sodium dodecylbenzene sulfonate is used as an external anionic surfactant during the dispersion process. The dispersion has a solid content of about 48 wt. % and had a number average particle size of about 0.4 micron. Likewise, this high solid aqueous polyurethane dispersion is substantially free of tertiary amines, and made under a VOC-free manufacturing process. Such a dispersion can be obtained commercially from The Dow Chemical Company (reference XUR-2005YM248279).

The specific concentration of aqueous polyurethane dispersion depends on the particular end use of the composition. It is preferred that the aqueous polyurethane dispersion be present in the personal care composition in an amount effective to enhance at least one of the following properties of the composition—water resistance, shine, matte, after-feel, appearance, color retention, or curl retention. In one embodiment, the aqueous polyurethane dispersion is present in an amount from about 0.02% to about 50% by weight of the composition. In one embodiment, the aqueous polyurethane dispersion is present in an amount from about 0.5% to about 10% by weight of the composition. In another embodiment, the aqueous polyurethane dispersion is present in an amount from about 5% to about 10% by weight of the composition. This includes all subcombinations of ranges, as well as greater than 5%, greater than 6%, greater than 7%, greater than 8%, and greater than 9%, by weight of the composition.

In some embodiments, the personal care composition includes an emollient. The emollient may be at least one of moisturizer, conditioner, oil, or other fatty substance. For example, when the composition is in an emulsion form, it comprises at least one oily phase that contains at least one oil, especially a cosmetically acceptable oil. The term “oil” means a fatty substance that is liquid at room temperature.

Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID 350 silicone fluid (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

In one embodiment, the emollient is present in an amount from about 0.05% to about 20% by weight of the composition. Preferably, the emollient is present in an amount from about 0.1% to about 10% by weight of the composition.

In some embodiments, the personal care composition includes an emulsifier or a surfactant. Suitable emulsifiers are selected from amphoteric, anionic, cationic and nonionic emulsifiers, used alone or as a mixture. Anionic surfactants include soaps or salts of fatty acids, alkyl sulfates, alkyl ether sulfates, alpha-olefin sulfonates, alkyl aryl sulfonates, sarcosinates, alkyl glucose esters or their alkoxylates, and in particular sodium lauryl sulfate, ammonium lauryl sulfate, triethanolamine lauryl sulfate, sodium laureth sulfate, isethionates, and triethanolamine stearate. Nonionic surfactants include methyl glucose stearates or their ethoxylates, alkyl polyglucosides, and glycerol monostearate, fatty acid alkanol amides, alkyl aryl polyglycol ether, polyglycol ethers and in particular cocoyl diethanolamide, nonoxynol-7 and octoxynol-9; cationics including alkyl trimethyl ammonium salts, quaternized amides of ethylene diamine, alkyl pyridinium salts and in particular cetrimonium chloride, stearalkonium chloride and cetyl pyridinium chloride; and amphoterics including alkyl.beta.-aminopropionates, betaines, alkyl imidazolines and in particular cocamidopropyl betaine and caproam phocarboxy propionate. Polymeric cationic emulsifiers that include hydrophobic moieties are preferred, examples of which include polyquaternium-24 and polyquatemium 67 (SOFTCAT™), available from The Dow Chemical Company.

Emulsions free of emulsifying surfactants or comprising less than 0.5% of emulsifying surfactants relative to the total weight of the composition may also be prepared, by using suitable compounds, for example polymers having emulsifying properties, such as CARBOPOL 1342 polymer (Noveon), PEMULEN polymer (Noveon), SEPIGEL 305 polyacrylamide/C13-C14 isoparaffin/laureth-7 (Seppic), particles of ionic or nonionic polymers, particles of anionic polymer such as, isophthalic acid, sulfoisophthalic acid polymers, and phthalate/sulfoisophthalate/glycol copolymers (for example diethylene glycol/phthalate/isophthalate/1,4-cyclohexanedimethanol sold under the names Eastman AQ diglycol/CHDM/isophthalates/SIP copolymer (AQ35S, AQ38S, AQ55S and/or AQ48 Ultra, from Eastman Chemical). Emulsifier-free emulsions stabilized with silicone particles or metal oxide particles such as TiO₂ or the like may also be prepared.

The surfactant may be present in an amount from about 0.01% to about 10% by weight of the composition. In one embodiment, the surfactant is present in an amount from about 0.1% to about 5% by weight of the composition.

In some embodiments, the personal care composition includes a thickener. Examples of thickeners include polymers, for example, modified or unmodified carboxyvinyl polymers, such as the products sold under the names CARBOPOL and PEMULEN (INCI name: Acrylates/C10-30 alkyl acrylate crosspolymer; available from Noveon), polyacrylates and polymethacrylates, such as the products sold under the names LUBRAJEL and NORGEL (from Guardian) or HISPAGEL (from Hispano Chimica), polyacrylamides, 2-acrylamido-2-methylpropanesulfonic acid polymers and copolymers, which are optionally crosslinked and/or neutralized, for instance the poly(2-acrylamido-2-methylpropane-sulfonic acid) sold by Clariant (INCI name: ammonium polyacryldimethyltauramide), emulsified crosslinked anionic copolymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate copolymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose, associative polymers, for instance associative polyurethanes, copolymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Hüls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Röhm & Haas). One preferred thickener is METHOCEL hydroxypropyl methylcellulose, available from The Dow Chemical Company.

In one embodiment, the thickener is present in an amount from about 0.01% to about 10% by weight of the composition. In one embodiment, the thickener is present in an amount from about 0.1% to about 2% by weight of the composition.

The personal care composition also comprises a suitable carrier, or mixtures of carriers. The type of carrier depends on the particular end use of the composition. Illustrative carriers include, for example, water, such as deionized or distilled water, emulsions, such as oil-in-water or water-in-oil emulsions, alcohols, such as ethanol, isopropanol or the like, glycols, such as propylene glycol, glycerine or the like, or combinations thereof. A preferred carrier is deionized water.

In one embodiment, the personal care compositions of the present invention further comprise an active ingredient selected from skin care actives, nail care actives, or hair care actives. Actives include sunscreens, skin colorants, drug substances (such as anti-inflammatory agents, antibiotics, topical anesthetics, antimycotics, keratolytics, and the like), skin protectants, conditioners, humectants, and ultraviolet radiation absorbers. Examples of sunscreens include paraminobenzoic acid, avobenzone, cinoxate, dioxybenzone, homosalate, menthyl anthranilate, octocrylene, octyl methoxycinnamate, octyl salicylate, oxybenzone, padimate 0, phenylbenzimidazole sulfonic acid, sulisobenzone, trolamine salicylate, titanium dioxide and zinc oxide, diethanolamine methoxycinnamate, digalloy trioleate, ethyl dihydroxypropyl PABA, glyceryl aminobenzoate, lawsone with dihydroxy acetone, and red petrolatum.

In one embodiment, the personal care compositions of the present invention further comprise at least one additional ingredient. Optional ingredients include any suitable substance for personal care compositions, for example, colorants, preservatives, pH adjustors, reducing agents, fragrances, foaming agents, tanning agents, depilatory agents, flavors, astringents, antiseptics, deodorants, antiperspirants, insect repellants, bleaches and lighteners, anti-dandruff agents, adhesives, polishes, strengtheners, fillers, barrier materials, and biocides.

Colorants include pigments, which are used especially in make-up, including metal oxide pigments, titanium dioxide, optionally surface-treated, zirconium oxide or cerium oxide, zinc oxide, iron oxide (black, yellow or red), chromium oxide, manganese violet, ultramarine blue, chromium hydrate and ferric blue, carbon black, pigments of barium, strontium, calcium or aluminum (for example D&C or FD&C), cochineal carmine, mica coated with titanium or with bismuth oxychloride, titanium mica with iron oxides, titanium mica with, especially, ferric blue or chromium oxide, titanium mica with an organic pigment, nacreous pigments based on bismuth oxychloride, goniochromatic pigments, for example pigments with a multilayer interference structure, reflective pigments, for example particles with a silver-coated glass substrate, glass substrate coated with nickel/chromium/molybdenum alloy, glass substrate coated with brown iron oxide, particles comprising a stack of at least two polymer layers, for instance MIRROR GLITTER (from 3M).

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, and sodium hydroxide. In a preferred embodiment, the pH adjustor is aminomethyl propanol, triethanolamine, L-arginine, tromethamine, PEG-15 cocamine, diisopropanolamine, triisopropanolamine, or tetrahydroxypropyl ethylenediamine. In a particularly preferred embodiment, the pH adjustor is amino methyl propanol, Aminomethyl propanol is available under the tradename AMP-ULTRA from Angus Chemical Company. In one embodiment, the pH adjustor is present in an amount from about 0.01% to about 1% by weight of the composition. In one embodiment, the pH adjustor is present in an amount from about 0.1% to about 0.5% by weight of the composition.

Reducing agents include ammonium thioglycolate, hydroquinone and sodium thioglycolate.

Fragrances include any component which provides a pleasant scent. Fragrances are generally aldehydes or ketones, and often oils obtained by extraction of natural substances or synthetically produced. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

The amount of optional ingredients effective for achieving the desired property provided by such ingredients can be readily determined by one skilled in the art.

### EXAMPLES

The following examples are for illustrative purposes only and are not intended to limit the scope of the present invention. All percentages are by weight unless otherwise specified.

### Example 1

An exemplary sunscreen lotion according to the present invention contains the components recited in TABLE 1:

TABLE 1

| Seq. | Component | Tradename | % |
|---|---|---|---|
| A | Water | | 62.18 |
| | Hydroxypropyl | METHOCEL E4M Hydroxypropyl | 5.0 |
| | Methylcellulose | Methylcellulose 2% sol. | |
| | | (The Dow Chemical Company) | |
| | Disodium EDTA | VERSENE NA Disodium EDTA | 0.05 |
| | | (The Dow Chemical Company) | |
| | Polysorbate 20 | TWEEN 20 | 0.4 |
| | | (Atlas Chemical Industries) | |
| | Propylene glycol, | NIPAGUARD | 0.8 |
| | diazolidinyl urea, methyl | (Clariant) | |
| | paraben, propyl paraben | | |
| B | Octyl methoxycinnamate | ESCALOL 557 | 7.50 |
| | | (ISP (International Specialty Products)) | |
| | Benzophenone-3 | UVINUL M40 (BASF) | 6.0 |
| | 2-ethylhexyl salicylate | DERMOBLOCK OS (Alzo) | 5.0 |
| | C₁₂₋₁₅ alkyl benzoate | FINSOLV TN (Finetex Inc.) | 5.0 |
| | Acrylates/C₁₀₋₃₀ Alkyl | PEMULEN TR-1 (Noveon) | 0.25 |
| | Acrylate Crosspolymer | | |
| | Acrylates/C₁₀₋₃₀ Alkyl | CARBOPOL ETD 2020 (Noveon) | 0.10 |
| | Acrylate Crosspolymer | | |
| C | Aminomethyl Propanol | AMP-ULTRA (Angus Chemical | 0.22 |
| | | Company) | |
| D | Aqueous polyurethane | XUR-2005YM248279, about 50% solid | 7.5 |
| | dispersion based on IPDI | (The Dow Chemical Company) | |
| | and an aliphatic polyester polyol | | |

Part A ingredients are combined and mixed until homogenous. In a separate vessel, the octyl methoxycinnamate, benzophenone-3,2-ethylhexyl salicylate, and C₁₂₋₁₅ alkyl benzoate ingredients of Part B are combined and mixed using moderate agitation with heat until the oxybenzone dissolves. The mixture is cooled to 45° C. and the acrylates are added with continuing mixing. Parts A and B are added with vigorous agitation until a smooth, non-grainy dispersion is achieved.

Part C is added to the dispersion, and mixing continues at a moderate rate, for example, 800-1200 rpm on a propeller mixer, for approximately an hour or until well mixed.

Part D is added to the mixture and mixing continues until the lotion is uniform. If desired, the preservative can be added at this stage instead of as a part of Part A.

### Example 2

### Comparative

Comparative sunscreen lotions were prepared using a protocol substantially similar to that described in Example 1, but containing the components recited in TABLE 2:

TABLE 2

| Seq. | Component | Tradename | Sample 1% | Sample 2% |
|---|---|---|---|---|
| A | Water | | 69.68 | 62.18 |
| | Hydroxypropyl | METHOCEL E4M Hydroxypropyl | 5.0 | 5.0 |
| | Methylcellulose | Methylcellulose 2% sol. | | |
| | | (The Dow Chemical Company) | | |
| | Disodium EDTA | VERSENE NA Disodium EDTA | 0.05 | 0.05 |
| | | (The Dow Chemical Company) | | |
| | Polysorbate 20 | TWEEN 20 | 0.4 | 0.4 |
| | | (Atlas Chemical Industries) | | |
| | Propylene glycol, | NIPAGUARD | 0.8 | 0.8 |
| | diazolidinyl urea, | (Clariant) | | |
| | methyl paraben, | | | |
| | propyl paraben | | | |
| B | Octyl | ESCALOL 557 | 7.50 | 7.50 |
| | methoxycinnamate | (ISP (International Specialty | | |
| | | Products)) | | |
| | Benzophenone-3 | UVINUL M40 | 6.0 | 6.00 |
| | | (BASF) | | |
| | 2-ethylhexyl | DERMOBLOCK OS | 5.0 | 5.00 |
| | salicylate | (Alzo) | | |
| | C₁₂₋₁₅ alkyl benzoate | FINSOLV TN | 5.0 | 5.00 |
| | | (Finetex Inc.) | | |
| | Acrylates/C₁₀₋₃₀ | PEMULEN TR-1 | 0.25 | 0.25 |
| | Alkyl Acrylate | (Noveon) | | |
| | Crosspolymer | | | |
| | Acrylates/C₁₀₋₃₀ | CARBOPOL ETD 2020 | 0.10 | 0.10 |
| | Alkyl Acrylate | (Noveon) | | |
| | Crosspolymer | | | |
| C | Aminomethyl | AMP-ULTRA | 0.22 | 0.22 |
| | Propanol | (Angus Chemical Company) | | |
| D | PPG- | AVALURE UR 450 | — | 7.5 |
| | 17/IPDI/DMPA | (Noveon) | | |
| | copolymer | | | |

### Example 3

In the water resistance test, 0.05 ml of the sunscreen lotion is applied to a simulated rough skin surface of a VITRO-SKIN substrate (available from IMS Inc.) with a syringe. The sample then is distributed evenly over the entire surface area by using a glove finger and allowed to dry for 30 minutes.

The dried sample is then soaked with 35 grams of deionized water in a 80 ml vial and shaken for 20 minutes at maximum agitation with a shaker. The remaining sunscreen agent on the skin is extracted with 50% isopropyl alcohol solution. Similarly, the sun screen agent in the wash water is extracted (with 50% isopropyl alcohol solution). The amount of sunscreen agent in the extracted solutions is measured by a UV spectrophotometer.

A sunscreen lotion according to the present invention was prepared using components and protocol substantially similar to that described in Example 1. This lotion was tested against the comparative samples of Example 2 for water resistance using a water resistance testing protocol substantially similar to that described above.

The sunscreen lotion according to the present invention lost only 3.18% in the rinse. By comparison, comparative Sample 1 lost 81.54% and comparative Sample 2 lost 4.31%. Moreover, Sample 2 has tertiary amines, which is not desirable for a personal care composition.

### Example 4

An exemplary foundation according to the present invention contains the components recited in TABLE 3:

TABLE 3

| Seq. | Component | Tradename | % |
|---|---|---|---|
| A | PEG-6 Stearate and PEG-20 | TEFOSE 1500 | 5.0 |
| | Stearate | (Gattefosse') | |
| | Isostearyl Isostearate | (Gattefosse') | 4.0 |
| | Cetearyl Alcohol | LANETTE O | 1.7 |
| | | (Cognis) | |
| | Isononyl Isononanate | WICKENOL 151 | 5.0 |
| | | (Alzo) | |
| | D5 and D6 | DOW CORNING 345 | 10.0 |
| | Decamethylcyclopentasiloxane | (Dow Corning) | |
| B | Water (Deionized) | | 43.06 |
| | Acrylates/C₁₀₋₃₀ Alkyl | CARBOPOL ETD 2020 | 0.10 |
| | Acrylate Crosspolymer | (Noveon) | |
| | Propylene Glycol | (The Dow Chemical Company) | 3.0 |
| C | Aminomethyl Propanol | AMP-ULTRA | 0.12 |
| | | (Angus Chemical Company) | |
| D | Aqueous polyurethane | XUR-2005YM248279, about 50% | 7.0 |
| | dispersion based on IPDI and | solid | |
| | an aliphatic polyester polyol | (The Dow Chemical Company) | |
| E | 46.2% TiO₂ | DDS TiO₂ | 15.38 |
| | | (The Dow Chemical Company) | |
| | 28.8% yellow | DDS Yellow I/O | 3.33 |
| | | (The Dow Chemical Company) | |
| | 42% red | DDS Red I/O | 0.95 |
| | | (The Dow Chemical Company) | |
| | 39.6% black | DDS Black I/O | 0.35 |
| | | (The Dow Chemical Company) | |
| F | Propylene glycol, diazolidinyl | NIPAGUARD | 1.0 |
| | urea, methyl paraben, propyl paraben | (Clariant) | |

Part A ingredients, with the exception of the cyclomethicone, are combined and heated to 85° C. The cyclomethicone is then added to the melted oil phase.

To prepare the Part B ingredients, CARBOPOL ETD 2020 is added to deionized water and heated to 60° C. This is mixed until polymer disperses.

To make an emulsion, Part A and Part B are combined under mixing at ˜600 rpm. Then, the mixture is neutralized with AMP (Part C) and mixed at higher speed. The aqueous polyurethane dispersion is added and mixed for about five minutes. The pigment dispersion is added to the emulsion and mixed at ˜4200 rpm for about ten minutes. Then Part F is added to the emulsion. Water loss is compensated for and the pH is adjusted with AMP-95 to 7.5. Cooling is completed with gentle stiffing.

### Example 5

### Comparative

Comparative foundations containing the components recited in TABLE 4 were prepared using a protocol substantially similar to that described in Example 4:

TABLE 4

| Seq. | Component | Tradename | % |
|---|---|---|---|
| A | PEG-6 Stearate and PEG-20 Stearate | TEFOSE 1500 | 5.0 |
| | | (Gattefosse') | |
| | Isostearyl Isostearate | (Gattefosse') | 4.0 |
| | Cetearyl Alcohol | LANETTE O (Cognis) | 1.7 |
| | Isononyl Isononanate | WICKENOL 151 (Alzo) | 5.0 |
| | D5 and D6 | DOW CORNING 345 | 10.0 |
| | Decamethylcyclopentasiloxane | (Dow Corning) | |
| B | Water (Deionized) | | 43.06 |
| | Acrylates/C₁₀₋₃₀ Alkyl | CARBOPOL ETD 2020 | 0.10 |
| | Acrylate Crosspolymer | (Noveon) | |
| | Propylene Glycol | (The Dow Chemical Company) | 3.0 |
| C | Aminomethyl Propanol | AMP-ULTRA | 0.12 |
| | | (Angus Chemical Company) | |
| D | PPG-17/IPDI/DMPA | AVALURE UR 450 | 7.0 |
| | copolymer | (Noveon) | |
| E | 46.2% TiO₂ | DDS TiO₂ | 15.38 |
| | | (The Dow Chemical Company) | |
| | 28.8% yellow | DDS Yellow I/O | 3.33 |
| | | (The Dow Chemical Company) | |
| | 42% red | DDS Red I/O | 0.95 |
| | | (The Dow Chemical Company) | |
| | 39.6% black | DDS Black I/O | 0.35 |
| | | (The Dow Chemical Company) | |
| F | Propylene glycol, diazolidinyl urea, methyl | NIPAGUARD | 1.0 |
| | paraben, propyl paraben | (Clariant) | |

### Example 6

A liquid foundation of the present invention was prepared using components and protocol substantially similar to that described in Example 4. This foundation was tested against the comparative sample of Example 5 for sensory properties.

Ten panelists applied 0.05 g of each sample to a designated area on their right or left forearms. Initially, each sample was evaluated for ease of blending, play time, evenness of color deposit, coverage, speed of adsorbtion, shine, matte, skin moistness, heaviness, amount of grease, amount of tack, quickness of drying, overall skin feel, and overall appearance. The evaluation scale was 0-10, with 10 being the best. For these 14 criteria, the sample according to the present invention performed as well or better than the comparative sample of Example 5 with the exceptions of coverage (about 1 less) and greasiness (about 1 more). Among the parameters where the sample according to the present invention performed better than the comparative sample of Example 5, were the important parameters of overall appearance and overall feel.

After 2 hours, each sample was again evaluated, this time for coverage, evenness of coverage, shine, matte, skin moistness, heaviness, and overall appearance. The evaluation scale was 0-10, with 10 being the best. For these criteria, the sample according to the present invention performed better than the comparative sample of Example 5 without exception, coverage being the closest to equal. Overall appearance was significantly better for the sample according to the present invention.

### Example 7

A hair gel of the present invention contains the components recited in TABLE 5:

TABLE 5

| Component | Tradename | % |
|---|---|---|
| Carbomer | CARBOMER 940 (Noveon) | 0.75 |
| Propylene Glycol | (Eastman Chemical) | 1.5 |
| Aminomethyl Propanol | AMP-ULTRA (Angus Chemical | 0.75 |
| | Company) | |
| Aqueous polyurethane | XUR-2005YM248279, about 50% | 6.0 |
| dispersion based on IPDI and | solid (The Dow Chemical | |
| an aliphatic polyester polyol | Company) | |
| DMDM Hydantoin | GLYDANT (Lonza) | 0.4 |
| Deionized Water | | 90.6 |

CARBOMER 940 is dusted into water with rapid agitation and mixed until fully hydrated. The other ingredients are combined in order and mixed using the appropriate agitation speed like 500 rpm at room temperature.

### Example 8

### Comparative

Comparative hair gels containing the components recited in TABLE 6 were prepared using a protocol substantially similar to that described in Example 7:

TABLE 6

| | | Sam- | Sam- |
|---|---|---|---|
| Component | Tradename | ple 1% | ple 2% |
| Carbomer | CARBOMER 940 (Noveon) | 0.75 | 0.75 |
| Propylene Glycol | (Eastman Chemical) | 1.5 | 1.5 |
| Aminomethyl | AMP-ULTRA (Angus | 0.75 | 0.75 |
| Propanol | Chemical Company) | | |
| Polyvinylpyrrolidone | PVP K-30 (ISP (International | 3.0 | — |
| (PVP) | Specialty Products)) | | |
| PPG-17/IPDI/DMPA | AVALURE UR 450 (Noveon) | — | 6.0 |
| copolymer | | | |
| DMDM Hydantoin | GLYDANT (Lonza) | 0.4 | 0.4 |
| Deionized Water | | 93.6 | 90.6 |

### Example 9

A hair gel of the present invention was prepared using components and protocol substantially similar to that described in Example 7. This hair gel was tested against the comparative samples of Example 8 for hair stiffness and curl retention.

The stiffness of European brown hair treated with the above-described hair styling gels was measured by the three point bending mode with a DIA-STRON MTT 170 at room temperature and 50% relative humidity. The hair gel of the present invention demonstrated 604 gmf, substantially twice as much stiffness as Sample 1 (300 gmf) or Sample 2 (286 gmf).

The curl retention of European brown hair is measured using the Curl Droop protocol. In the Curl Droop protocol, hair swatches (5 g, 22 in.) are treated with 5 g hair gel, then curled with a 1 inch diameter curler, then dried at about 22° C. and 55% relative humidity overnight. The curl is carefully removed from the treated hair and slowly relaxed in a humidity chamber having controlled temperature (25° C.) and humidity (90% relative humidity), the length of hair swatches being measured over a 24 hour period.

The curl retention of European brown hair treated with the above-described hair styling gels was measured using a protocol substantially similar to the above-described Curl Droop protocol. Sample 1 retained less than thirty percent of the curl. Sample 2 retained less than ninety percent of the curl. However, the hair gel of the present invention retained about 95.5% of the curl, a significant breakthrough.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A personal care composition comprising:
a high solid aqueous polyurethane dispersion, substantially free of organic solvent and tertiary amines; and
at least one of a cosmetically acceptable emolient, surfactant, emulsifier, preservative, or rheology modifier; and
an active ingredient selected from skin care actives, nail care actives, or hair care actives.

2. (canceled)

3. The personal care composition of claim 1, further comprising at least one colorant, preservative, pH adjustor, propellant, reducing agent, fragrance, foaming agent, tanning agent, depilatory agent, flavor, astringent, antiseptic, deodorant, antiperspirant, insect repellant, bleach, lightener, anti-dandruff agent, adhesive, polish, strengthener, filler, barrier material, or biocide.

4. The personal care composition of claim 3, wherein the pH adjuster is aminomethyl propanol, triethanolamine, L-arginine, tromethamine, PEG-15 cocamine, diisopropanolamine, triisopropanolamine, or tetrahydroxypropyl ethylenediamine.

5. The personal care compositions of claim 4, wherein the pH adjustor is present in an amount from about 0.1% to about 1% by weight of the composition.

6. The personal care composition of claim 4, wherein the pH adjustor is present in an amount from about 0.01% to about 0.5% by weight of the composition.

7. The personal care composition of claim 1, wherein the aqueous polyurethane dispersion is based on isophorone diisocyanate and an aliphatic polyester polyol.

8. The personal care composition of claim 1, wherein the aqueous polyurethane dispersion is present in an amount from about 0.02% to about 50% by weight of the composition.

9. The personal care composition of claim 1, wherein the aqueous polyurethane dispersion is present in an amount from about 0.5% to about 10% by weight of the composition.

10. The personal care composition of claim 1, wherein the aqueous polyurethane dispersion is present in an amount from about 5% to about 10% by weight of the composition.

11. The personal care composition of claim 1, wherein the emollient is at least one of a moisturizer, oil, or other fatty substance.

12. The personal care composition of claim 1, wherein the emollient is present in an amount from about 0.5% to about 20% by weight of the composition.

13. The personal care composition of claim 1, wherein the emollient is present in an amount from about 0.1% to about 10% by weight of the composition.

14. The personal composition of claim 1, wherein the emulsifier is at least one of an anionic, nonionic, amphoteric, or cationic surfactant.

15. The personal care composition of claim 1, wherein the emulsifier or surfactant is present in an amount from about 0.01% to about 5% by weight of the composition.

16. The personal care composition of claim 1, wherein the emulsifier or surfactant is present in an amount from about 0.01% to about 5% by weight of the composition.

17. The personal care composition of claim 1, wherein the rheology modifier is at least one of a water soluble cellulose derivative, cellulose, starch, acrylate/C₁₀₋₃₀alkyl acrylate cross polymer, or water-soluble vinyl polymer.

18. The personal care composition of claim 1, wherein the rheology modifier is present in an amount from about 0.01% to about 10% by weight of the composition.

19. The personal composition of claim 1, wherein the rheology modifier is present in an amount from about 0.01% to about 2% by weight of the composition.

20. The personal composition of claim 1, wherein the personal care composition is a hair gel that demonstrates greater than 90% curl retention over 24 hours at 25° C. and 90% relative humidity.
